(19) United States
(12) Patent Application Publication
(10) Pub. No.: US 2005/0049241 A1
(43) Pub. Date: Mar. 3, 2005

(54) Pyridyl piperazinyl ureas

(76) Inventors: Nicholas I. Carruthers, Poway, CA (US); Chandravadan R. Shah, San Diego, CA (US); Devin M. Swanson, La Jolla, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/910,911

(22) Filed: Aug. 4, 2004

Related U.S. Application Data
(60) Provisional application No. 60/493,920, filed on Aug. 8, 2003.

(51) Int. Cl. A61K 31/55; A61K 31/496; C07D 43/02; A61K 31/551

(52) U.S. Cl. 514/218; 514/253.01; 544/360; 540/575

(57) ABSTRACT

There are disclosed novel pyridyl piperazinyl ureas that are potent antagonists of vanilloid receptor, VR1, and are useful for the treatment and/or prevention of i) acute or chronic pain or itch; ii) inflammation; iii) gastrointestinal and urinary tract disorders; and iv) tracheobronchial and diaphragmatic dysfunction in humans.

This invention is directed to novel vanilloid receptor VR1 agents. More particularly, this invention relates to novel pyridyl piperazinyl ureas that are potent antagonists of VR1, and are useful for the treatment and/or prevention of i) acute or chronic pain or itch; ii) inflammation; iii) gastrointestinal and urinary tract disorders; and iv) tracheobronchial and diaphragmatic dysfunction in humans.

### BACKGROUND OF THE INVENTION

Sensory information transmitted by primary afferent neurons confers on an organism the abilities to sample and react to its external environment, and the ability to maintain internal homeostasis. Some of this information is transmitted to conscious perception in a variety of modalities, including pain. Other information does not reach a conscious level but participates in lower-level reflexes. In general, pain and discomfort are variably perceived depending on the affected organ system, and may directly or indirectly include components of reflex responses such as smooth or skeletal muscle spasm, nausea, vomiting, and bladder or intestinal voiding urges.

Nociceptive neurons mediate the detection of tissue damage or of potentially harmful stimuli, as well as changes in the extracellular space that arise during inflammatory or ischemic conditions. Examples are heat, local tissue acidosis and tissue distension or stretch (Wall, PD and Melzack, R, Textbook of Pain, 1994, New York: Churchill Livingstone). Noxious chemical, thermal and mechanical stimuli excite peripheral nerve endings of small diameter sensory neurons (nociceptors) in sensory ganglia (e.g. dorsal root, nodose and trigeminal ganglia) and initiate signals that are perceived as pain and other forms of physical discomfort. Nociceptors transduce noxious stimuli into an electrical signal (membrane depolarization) that triggers orthodromic (forward propagation of) action potentials, which are conducted from the sensory sites to the CNS. Modulation of particular ion channels and receptors mediates the generator potential at the sensory neuron terminal. A subset or closely related class of these fibers also transmits the sensation of pruritus (itch).

Noxious chemical agents include both exogenous and endogenous substances, e.g. chemical mediators of inflammation. Under conditions of inflammation, nociceptor responses become sensitized. Enhanced nociceptor excitability greatly amplifies the response to the same stimulus.

Importantly, nociceptive fibers play an efferent role in inflammatory conditions as well as an afferent role. Stimulation of small fibers leads to antidromic (retrograde) discharge of neurotransmitters in addition to orthodromic conduction (axon reflex). Such release of neuroactive substances in the local environment of the nerve terminal plays an important part in the neurochemical cascades and cellular responses that contribute to tissue inflammation (Lin, Q.; Zou, X.; Willis, W. D. J. Neurophysiol. 2000, 84(5), 2695-2698; Sauer, S. K.; Bove, G. M.; Averbeck, B; Reeh, P. W. Neuroscience 1999, 92(1), 319-25). Inflammatory conditions may thus become vicious cycles of nociception leading to neurosecretion resulting in further inflammation and nociception.

Plant derived vanilloid compounds (e.g., capsaicin, the pungent component of hot chili peppers, and its ultrapotent analog, resiniferatoxin) are known to selectively depolarize nociceptors and elicit sensations of burning pain. These compounds are particularly irritating to mucosal surfaces, and, depending on dose and where applied, provoke cough, lacrimation, bronchorrhea, rhinorrhea, and elicit smooth muscle reflexes such as bronchoconstriction. Capsaicin thus mimics the action of physiological/endogenous stimuli that activate nociceptive/homeostatic afferent pathways. Recent advances in sensory biology have identified receptors for vanilloids, protons (i.e. acidic solutions) and heat. Because heightened activity of nociceptors contributes to unwanted pain, inflammatory conditions, thermoregulation, and control of smooth muscle tone and reflexes in human beings and animals, modulation of signaling in this pathway is important in palliation and remediation of these clinical syndromes (Caterina, M. J. Pain 2003, 105(1-2), 5-9).

Capsazepine, a VR1 antagonist, inhibits cough induced by capsaicin and citric acid in guinea pigs, in a manner consistent with a specific VR1 pharmacology (Lalloo, U. G.; Fox, A. J.; Belvisi, M. G.; Chung, K. F.; Barnes, P. J. J. Appl. Physiol. 1995, 79(4), 1082-7). This indicates that capsazepine may be useful as an antitussive agent.

Administration of VR1 antagonists has been demonstrated to have analgesic and anti-hyperalgesic properties in two preclinical pain models in both rat and guinea pig. In a complete Freund's adjuvant model of paw inflammation in the rat, administration of a VR1 antagonist compound has been shown to produce reversal of paw thermal hyperalgesia. In a partial sciatic nerve ligation model in the rat, administration of a VR1 antagonist has been shown to reduce mechanical hyperalgesia. These results have also been demonstrated in analogous guinea pig models of complete Freund's adjuvant induced paw inflammation and partial sciatic nerve ligation. The following reference discloses an in vitro characterization of BCTC as a VR1 antagonist: Valenzano, K. J.; Grant, E. R.; Wu, G.; Hachicha, M.; Schmid, L.; Tafesse, L.; Sun, Q.; Rotshteyn, Y.; Francis, J.; Limberis, J.; Malik, S.; Whittemore, E. R.; Hodges D. J. Pharmacol. Exp. Ther. 2003, 306(1), 377-86. The following reference discloses preclinical results of BCTC in two rat pain models: Pomonis. J. D.; Harrison, J. E.; Mark, L.; Bristol, D. R.; Valenzano, K. J.; Walker, K. J. Pharmacol. Exp. Ther. 2003, 306(1), 387-93. The following reference discloses preclinical results of the VR1 antagonist capsazepine in guinea pig pain models, wherein capsazepine reverses mechanical hyperalgesia in models of inflammatory and neuropathic pain: Walker, K. M.; Urban, L.; Medhurst, S. J.; Patel, S.; Panesar, M.; Fox, A. J.; McIntyre, P. J. Pharmacol. Exp. Ther. 2003, 304(1), 56-62.

In WO 02/08221 there is disclosed VR1 antagonists that are certain dipyridyl piperazinyl ureas. Particular reference is made to published claims 42 and 43 and to examples 93, 94 and 98.

### SUMMARY OF THE INVENTION

There are provided by the present invention VR1 antagonists which have the general formula: 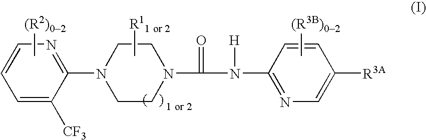
wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═.>NH or >N(C₁₋₄alkyl) and optionally having one unsaturated bond in the ring),
   where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONR^{a}R^{b},
   or alternatively,
   two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R² is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R^{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl,
   where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{y})R^{z}, —(C═O)N(R^{y})R^{z}, —(N—R^{t})COR^{t}, —(N—R^{t})SO₂C₁₋₆alkyl, —(C═O)C₁₋₆alkyl, —(C═O)phenyl, —(S═(O)₀₋₂)-C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{p})R^{q}, —(N—R^{s})COR^{s} (wherein R^{s} is —H or —C₁₋₆alkyl), —(N—R^{s})SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q}, —(C═O)N(R^{p})R^{q}, —(N—R^{s})COR^{s}, —(N—R^{s})SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl;
   or a stereoisomer or pharmaceutically acceptable salt, ester, amide or prodrug thereof.

### DETAILED DESCRIPTION OF THE INVENTION

Preferably, R¹ is selected from the group consisting of: —H, methyl, ethyl, n-propyl, i-propyl, n-butyl, i-butyl, t-butyl, n-pentyl, pent-2-yl, hexyl, hex-2-yl, ethenyl, allyl, ethynyl, prop-2-ynyl, cyclopentyl, cyclohexyl, cycloheptyl, trifluoromethyl, pentafluoroethyl, septafluoro-n-propyl, septafluoro-1-propyl, nonafluoro-n-butyl, nonafluoro-1-butyl, nonafluoro-t-butyl, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl and homopiperidin-1-yl,

wherein said methyl, ethyl, n-propyl, i-propyl, n-butyl, i-butyl, t-butyl, n-pentyl, pent-2-yl, hexyl, hex-2-yl, ethenyl, allyl, ethynyl and prop-2-ynyl primary substituents, are optionally mono-substituted with a substituent selected from the group consisting of —F, —Cl, —Br, —I, cyclopentyl, cyclohexyl, -cycloheptyl, trifluoromethyl, pentafluoroethyl, septafluoro-n-propyl, septafluoro-1-propyl, nonafluoro-n-butyl, nonafluoro-1-butyl, nonafluoro-t-butyl, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —(C═O)CH₃, —(C═O)CH₂CH₃, —(C═O)CH₂CH₂CH₃, —(C═O)CH(CH₃)₂, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl and —(C═O)homopiperidin-1-yl.

More preferably, R¹ is selected from the group consisting of: —H, methyl, ethyl, n-propyl, i-propyl, n-butyl, i-butyl and t-butyl, optionally mono-substituted with halo. Most preferably, R¹ is —H or methyl. The preferred R¹ is attached to a carbon atom attached to the urea nitrogen. Of course, where R¹ is other than hydrogen, a stereocenter is obtained. Compounds having either the R or S configuration at this stereocenter may be purified.

Where two R¹ are taken together to form a bridging group, the preferred bridging group is —CH₂— or —CH₂CH₂—. Preferably, carbon ring members are bridged that are separated by two ring members.

Preferably, R² are nonexistent or are independently selected from the group consisting of: methyl, ethyl, n-propyl, i-propyl, n-butyl, i-butyl, t-butyl, n-pentyl, pent-2-yl, hexyl, hex-2-yl, ethenyl, allyl, ethynyl, prop-2-ynyl, phenyl, —O-methyl, —O-ethyl, —O-n-propyl, —O-i-propyl, —O-n-butyl, —O-i-butyl, —O-t-butyl, —O-n-pentyl, —Opent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2, 3 or 4-yl, tetrahydrothiopyran-2, 3 or 4-yl, piperidin-2, 3 or 4-yl, N(C₁₋₄alkyl)piperidin-2, 3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-i-propyl, nonafluoro-n-butyl, nonafluoro-i-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl,

wherein said methyl, ethyl, n-propyl, i-propyl, n-butyl, i-butyl, t-butyl, n-pentyl, pent-2-yl, hexyl, hex-2-yl, ethenyl, allyl, ethynyl and prop-2-ynyl primary substituents, are optionally mono-substituted with a substituent selected from the group consisting of phenyl, —O-methyl, —O-ethyl, —O-n-propyl, —O-i-propyl, —O-n-butyl, —O-i-butyl, —O-t-butyl, —O-n-pentyl, —O-pent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2, 3 or 4-yl, tetrahydrothiopyran-2, 3 or 4-yl, piperidin-2, 3 or 4-yl, N(C₁₋₄alkyl)piperidin-2,3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-i-propyl, nonafluoro-n-butyl, nonafluoro-i-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl. More preferably, R² are nonexistent or are selected from the group consisting of —NO₂, —CF₃, —Cl, —F, —CH₃, —CN, —NH₂, —N(CH₃)₂, —OCH₃, tetrahydropyranyl, —CN, —NO₂ and —SO₂NH₂.

Preferably, R^{3A} and R^{3B} are independently selected from the group consisting of: methyl, ethyl, n-propyl, i-propyl, n-butyl, i-butyl, t-butyl, n-pentyl, pent-2-yl, hexyl, hex-2-yl, ethenyl, allyl, ethynyl, prop-2-ynyl, phenyl, —O-methyl, —O-ethyl, —O-n-propyl, —O-1-propyl, —O-n-butyl, —O-1-butyl, —O-t-butyl, —O-n-pentyl, —O-pent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2, 3 or 4-yl, tetrahydrothiopyran-2, 3 or 4-yl, piperidin-2, 3 or 4-yl, N(C₁₋₄alkyl)piperidin-2, 3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-1-propyl, nonafluoro-n-butyl, nonafluoro-1-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl,

wherein said methyl, ethyl, n-propyl, i-propyl, n-butyl, i-butyl, t-butyl, n-pentyl, pent-2-yl, hexyl, hex-2-yl, ethenyl, allyl, ethynyl and prop-2-ynyl primary substituents, are optionally mono-substituted with a substituent selected from the group consisting of phenyl, —O-methyl, —O-ethyl, —O-n-propyl, —O-i-propyl, —O-n-butyl, —O-i-butyl, —O-t-butyl, —O-n-pentyl, —O-pent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2, 3 or 4-yl, tetrahydrothiopyran-2, 3 or 4-yl, piperidin-2, 3 or 4-yl, N(C₁₋₄alkyl)piperidin-2,3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-i-propyl, nonafluoro-n-butyl, nonafluoro-i-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl. More preferably, R^{3A} and R^{3B} are independently selected from the group consisting of —CF₃, —OCF₃, butyl, i-propyl, t-butyl, cyclohexyl, cyclopentyl, tetrahydropyranyl, piperidin-1-yl, 1-cyano-1-methylethyl, 2-methoxy-1,1-dimethylethyl, bromo, chloro, fluoro, iodo, methyl, methoxy, nitro, benzyl, 1-trifluoromethylethenyl, 1-trifluoromethylethyl, but-2-yl, benzoyl, nonafluoro-t-butyl and septafluoro-1-propyl. Most preferably, R^{3A} is trifluoromethyl. It is also preferred that R^{3B} is nonexistent.

The “pharmaceutically acceptable salts and esters thereof” refer to those salt and ester forms of the compounds of the present invention which would be apparent to the pharmaceutical chemist, i.e., those which are non-toxic and which would favorably affect the pharmacokinetic properties of said compounds of the present invention. Those compounds having favorable pharmacokinetic properties would be apparent to the pharmaceutical chemist, i.e., those which are non-toxic and which possess such pharmacokinetic properties to provide sufficient palatability, absorption, distribution, metabolism and excretion. Other factors, more practical in nature, which are also important in the selection, are cost of raw materials, ease of crystallization, yield, stability, hygroscopicity, and flowability of the resulting bulk drug. In addition, acceptable salts of carboxylates include sodium, potassium, calcium and magnesium. Examples of suitable cationic salts include hydrobromic, hydroiodic, hydrochloric, perchloric, sulfuric, maleic, fumaric, malic, tartatic, citric, benzoic, mandelic, methanesulfonic, hydroethanesulfonic, benzenesulfonic, oxalic, pamoic, 2-naphthalenesulfonic, p-toluenesulfonic, cyclohexanesulfamic and saccharic. Examples of suitable esters include such esters where one or more carboxyl substituents is replaced with p-methoxybenzyloxycarbonyl, 2,4,6-trimethylbenzyloxycarbonyl, 9-anthryloxycarbonyl, CH₃SC H₂COO—, tetrahydrofur-2-yloxycarbonyl, tetrahydropyran-2-yloxycarbonyl, fur-2-uloxycarbonyl, benzoylmethoxycarbonyl, p-nitrobenzyloxycarbonyl, 4-pyridylmethoxycarbonyl, 2,2,2-trichloroethoxycarbonyl, 2,2,2-tribromoethoxycarbonyl, t-butyloxycarbonyl, t-amyloxycarbonyl, diphenylmethoxycarbonyl, triphenylmethoxycarbonyl, adamantyloxycarbonyl, 2-benzyloxyphenyloxycarbonyl, 4-methylthiophenyloxycarbonyl, or tetrahydropyran-2-yloxycarbonyl.

Preferred compounds of the present invention are selected from the group consisting of:

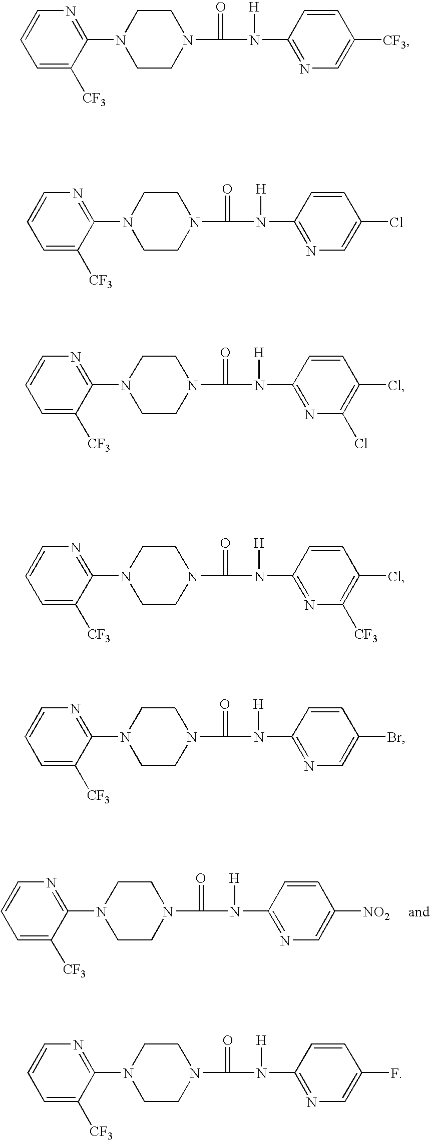

In a particular embodiment, a preferred compound of the present invention is selected from

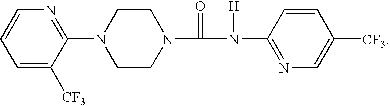

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

The pyridyl piperazinyl ureas are prepared by the synthetic method outlined as follows.

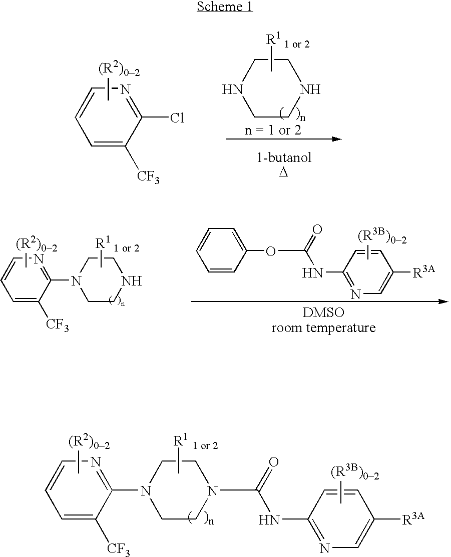

Compounds of the present invention may be prepared according to Scheme 1 whereby an appropriately substituted 2-halopyridine, preferably a 2-chloro or 2-bromopyridine is treated with a piperazine or homopiperazine in a solvent at a suitable temperature to afford a pyridyl piperazine. In a preferred embodiment, the piperazine or homopiperazine is used in excess, in a solvent at elevated temperature. More preferably, the piperazine or homopiperazine is used in an alcohol solvent, preferably 1-butanol or the like, and the reaction effected at the boiling point of the selected solvent. The pyridyl piperazine or pyridyl homopiperazine is then treated with an amino-pyridine carbamate, preferably a phenyl carbamate in a solvent, preferably DMSO or the like, at room temperature to afford compounds of formula (I).

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3rd Edition, John Wiley & Sons, 1999. The protecting groups may be removed at a convenient subsequent stage using methods known from the art.

Even though the compounds of the present invention (including their pharmaceutically, acceptable salts and pharmaceutically acceptable solvates) can be administered alone, they will generally be administered in admixture with a pharmaceutical carrier, excipient or diluent selected with regard to the intended route of administration and standard pharmaceutical or veterinary practice. Thus, the present invention is directed to pharmaceutical and veterinary compositions comprising compounds of formula (I) and one or more pharmaceutically acceptable carriers, excipients or diluents.

Tablets or capsules of the compounds may be administered singly or two or more at a time, as appropriate. It is also possible to administer the compounds in sustained release formulations. Alternatively, the compounds of the general formula (I) can be administered by inhalation or in the form of a suppository or pessary, or they may be applied topically in the form of a lotion, solution, cream, ointment or dusting powder. An alternative means of transdermal administration is by use of a skin patch. For example, the compounds can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilizers and preservatives as may be required. For some applications, preferably the compositions are administered orally in the form of tablets containing excipients such as starch or lactose, or in capsules or ovules either alone or in admixture with excipients, or in the form of elixirs, solutions or suspensions containing flavoring or coloring agents. The compositions (as well as the compounds alone) can also be injected parenterally, for example intracavernosally, intravenously, intramuscularly, subcutaneously, epidurally, intrathecally, or intracerebroventricularly. In this case, the compositions will comprise a suitable carrier or diluent. For parenteral administration, the compositions are best used in the form of a sterile aqueous solution that may contain other substances, for example enough salts or monosaccharides to make the solution isotonic with blood. For buccal or sublingual administration the compositions may be administered in the form of tablets or lozenges, which can be formulated in a conventional manner.

By way of further example, pharmaceutical and veterinary compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate the major site of absorption. For parenteral administration, the carrier will usually consist of sterile water and other ingredients may be added to increase solubility or preservation. Injectable suspensions or solutions may also be prepared utilizing aqueous carriers along with appropriate additives.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those skilled in that art. To be administered in the form of a transdermal delivery system, the dosage administration will, of course, be continuous rather than intermittent throughout the dosage regimen.

A therapeutically effective amount for use of the instant compounds or a pharmaceutical composition thereof comprises a dose range of from about 0.001 mg to about 1,000 mg, in particular from about 0.1 mg to about 500 mg or, more particularly from about 1 mg to about 250 mg of active ingredient per day for an average (70 kg) human. For oral administration, a pharmaceutical composition is preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated.

It is also apparent to one skilled in the art that the therapeutically effective dose for active compounds of the invention or a pharmaceutical composition thereof will vary according to the desired effect. Therefore, optimal dosages to be administered may be readily determined and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

The invention also provides a pharmaceutical or veterinary pack or kit comprising one or more containers filled with one or more of the ingredients of the pharmaceutical and veterinary compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

As modulators of the vanilloid VR1 ion channel, the compounds of formula (I), are useful in methods for treating or preventing a disease or condition in a mammal which disease or condition is affected by the modulation of one or more vanilloid receptors. Such methods comprise administering to a mammal in need of such treatment or prevention a therapeutically effective amount of a compound, salt or solvate of formula (I). In particular, the compounds of formula (I) are useful in methods for preventing or treating: i) acute or chronic pain or itch; ii) inflammation; iii) gastrointestinal and urinary tract disorders; and iv) tracheobronchial and diaphragmatic dysfunction.

By way of example only, the compounds of formula (I) are useful for treating acute or chronic pain arising from conditions selected from the group consisting of: osteoarthritis, rotator cuff disorders, rheumatoid arthritis, inflammatory arthritis, fibromyalgia, cluster headache, migraine, headache, sinus headache, tension headache, toothache, burn, sunburn, dermatitis, psoriasis, eczema, insect sting or bite, bony fractures, ligamentous sprains, plantar fasciitis, costochondritis, tendonitis, bursitis, tennis elbow, pitcher's elbow, patellar tendonitis, repetitive strain injury, myofascial syndrome, muscle strain, myositis, temporomandibular joint disorder, stump pain, low back strain, neck strain, whiplash, bladder spasms, interstitial cystitis, urinary tract infection, urethral colic, renal colic, pharyngitis, cold sores, stomatitis, external otitis, otitis media, burning mouth syndrome, mucositis, esophageal pain, gastroesophageal reflux, pancreatitis, enteritis, irritable bowel disorder, inflammatory bowel disease, Crohn's disease, ulcerative colitis, diverticulosis, diverticulitis, hemorrhoids, anal fissures, proctitis, rectal pain, cholecystitis, labor, childbirth, intestinal gas, abdominal pain, menstrual cramps, pelvic pain, vulvodynia, vaginitis, testicular pain, pleurisy, pericarditis, contusions, abrasions, peripheral neuropathy, diabetic neuropathy, acute herpetic neuralgia, post-herpetic neuralgia, trigeminal neuralgia, glossopharyngeal neuralgia, atypical facial pain, causalgia, reflex sympathetic dystrophy, sciatica, cervical, thoracic or lumbar radiculopathy, brachial plexopathy, lumbar plexopathy, phantom limb pain, occipital neuralgia, intercostal neuralgia, supraorbital neuralgia, inguinal neuralgia, meralgia paresthetica, genitofemoral neuralgia, carpal tunnel syndrome, Morton's neuroma, post-mastectomy syndrome, post-thoracotomy syndrome, post-polio syndrome, Guillain-Barre syndrome, Raynaud's syndrome, coronary artery spasm (Printzmetal's or variant angina), esophageal spasm, osteolytic metastases, osteoblastic metastases, primary bone cancer, cancerous invasion of bone, visceral cancer pain, neuropathic cancer pain, Paget's disease and multiple myeloma. Similarly, the compounds of formula (I) are useful for the treatment of itching arising from dermatological or inflammatory conditions selected from the group consisting of: renal or hepatobiliary disorders, immunological disorders, medication reactions and unknown/idiopathic conditions.

By way of example only, the compounds of formula (I) are useful for treating inflammatory manifestations of diseases and conditions selected from the group consisting of: inflammatory bowel disease (ulcerative colitis and Crohn's disease) psoriasis and psoriatic arthritis, rheumatoid arthritis, myasthenia gravis, multiple sclerosis, scleroderma, glomerulonephritis, pancreatitis, inflammatory hepatitis, asthma, chronic obstructive pulmonary disease, allergic rhinitis, uveitis and cardiovascular manifestations of inflammation including atherosclerosis, myocarditis, pericarditis and vasculitis.

By way of example only, the compounds of formula (I) are useful for the treatment of gastrointestinal and urinary tract disorders selected from the group consisting of: nausea, vomiting, intestinal cramping, intestinal bloating, bladder spasms, urinary urgency, defecation urgency and urge incontinence.

By way of example only, the compounds of formula (I) are useful for the treatment of tracheobronchial and diaphragmatic dysfunction associated with conditions selected from the group consisting of: cough, asthma, bronchospasm, chronic obstructive pulmonary disease, chronic bronchitis, emphysema and hiccups (hiccoughs, singuitus).

### EXAMPLES

In order to illustrate the invention, the following examples are included. These examples do not limit the invention. They are only meant to suggest a method of practicing the invention. Those skilled in the art may find other methods of practicing the invention, which are obvious to them. However, those methods are deemed to be within the scope of this invention.

### Experimental

NMR spectra were obtained on a Bruker model DPX400 (400 MHz) spectrometer. The format of the ¹H NMR data below is: chemical shift in ppm down field of the tetramethylsilane reference (multiplicity, coupling constant J in Hz, integration).

Flash column chromatography was accomplished using an ISCO Foxy 200 system employing one of the following commercially available prepacked columns: Biotage 40S (SiO₂ 40 g), Biotage 40M (SiO₂ 90 g), Biotage 40L (SiO₂ 120 g), Biotage 65M (SiO₂ 300 g).

### Example 1

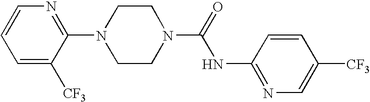 4-(3-Trifluoromethyl-pyridin-2-yl)-piperazine-1-carboxylic acid (5-trifluoromethyl-pyridin-2-yl)-amide

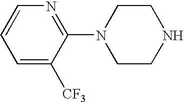

A. 1-(3-Trifluoromethyl-pyridin-2-yl)-piperazine. A solution of 2-chloro-3-trifluoromethylpyridine (10 g) and piperazine (47 g) in 1-butanol (400 mL) was heated to reflux. After 18 h the resulting mixture was concentrated under reduced pressure, then diluted with ethyl acetate (500 mL) and washed with 1 N sodium bicarbonate (200 mL). The organic layer was dried (Na₂SO₄), and the solvent was removed. Chromatography of the residue (SiO₂; 5-10% 2 M ammonia in methanol/dichloromethane) gave the title compound as a solid (10 g). mp: 38.8-42.9° C.

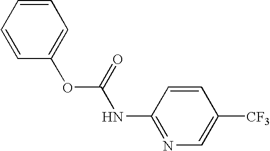

B. (5-Trifluoromethyl-pyridin-2-yl)-carbamic acid phenyl ester. A solution of 2-amino-5-triflouromethylpyridine (4 g) in tetrahydrofuran (50 mL) was cooled to 0° C. (ice bath) and stirred for 30 min. A solution of phenyl chloroformate (3.1 mL) in tetrahydrofuran (50 mL) was then added dropwise to the mixture via an addition funnel. After 18 h the reaction mixture was concentrated under reduced pressure, diluted with ethyl acetate (500 mL), and washed with 1 N sodium bicarbonate (250 mL). The organic layer was dried (Na₂SO₄), and the solvent was removed. Chromatography of the solid (SiO₂; 0-1% 2 M ammonia in methanol/dichloromethane) gave the title compound as a white solid (3 g). mp: 203.6-204.8° C.

C. 4-(3-Trifluoromethyl-pyridin-2-yl)-piperazine-1-carboxylic acid (5-trifluoromethyl-pyridin-2-yl)-amide. A solution of the product of step A (3 g) and the product of step B (3.7 g) in dimethylsulfoxide (40 mL) was stirred for 18 h. The reaction mixture was diluted with dichloromethane (500 mL) and washed with 1 N sodium hydroxide (2×200 mL) and water (3×200 mL). The organic layer was dried (Na₂SO₄), and the solvent was removed. Chromatography of the colored solid (SiO₂; 20-40% ethyl acetate/hexanes) gave the title compound as a white solid (4.3 g). mp: 89.4-89.8° C. ¹H NMR (400 MHz, CD₃OD): 8.53-8.50 (m, 2H), 8.06-8.03 (m, 1H), 8.00-7.94 (m, 2H), 7.23-7.19 (m, 1H), 3.74-3.71 (m, 4H), 3.30-3.27 (m, 4H). Elemental analysis: calculated for C₁₇H₁₅F₆N₅O, C, 48.69; H, 3.61; N, 16.70; found, C, 48.90; H, 3.61; N, 16.54.

### Comparative Example A

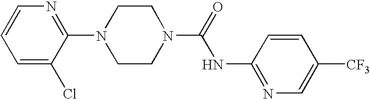 4-(3-chloro-pyridin-2-yl)-piperazine-1-carboxylic acid (5-trifluoromethyl-pyridin-2-yl)-amide

The title compound was made but is not part of the invention. It is compound 93 as disclosed in WO 02/08221.

### Biological Example

### Functional assay: block of capsaicin-induced Ca²⁺ influx

HEK293 cells were transfected with human VR1 cloned in pcDNA3.1zeo(+) using the Effectene non-liposomal lipid based transfection kit (Qiagen) (hVR1/HEK293). hVR1/HEK293 cells were routinely grown as monolayers under selection in zeocin (200 μg/ml; Invitrogen) in Dulbecco's Modified Eagle Medium (DMEM, Gibco BRL) supplemented with 10% fetal bovine serum, and penicillin/streptomycin (50 units/mL) in 5% CO₂ at 37° C. Cells were passaged frequently, every 3-5 days, to avoid acidic medium exposure. Cells were passaged without enzymes or Ca²⁺ chelators. Transfected cells were seeded onto poly-D-lysine coated black-walled 96-well plates (Biocoat; Becton Dickinson #354640) at about 40,000 cells per well and grown for at least 1 day in culture medium to near confluency. On the day of the experiment, media was manually removed using a 12-prong aspirator, incubated in 100 μL Fluo-3/AM (2 μM; Molecular Probes, Eugene, Oreg.) with Pluronic acid (0.04%; Molecular Probes, Eugene, Oreg.) for 1 hr at room temperature in the dark. After loading the cells, the dye solution was aspirated, 160 μL buffer was added to all wells, and intracellular Ca²⁺ levels were subsequently assayed using a Fluorometric Imaging Plate Reader (FLIPR™ instrument, Molecular Devices, CA) to simultaneously monitor Fluo-3 fluorescence in all wells (λ_{excitation}=488 nm, λₑₘᵢₛₛᵢₒₙ=540 nm). Antagonists were added on line (9-fold concentration in 20 μl added to 160 μL at a velocity of 20 μL/s) and fluorescence counts were captured every 3 sec for 3 min prior to agonist addition. Alternatively, compound was added to all wells using the Fluorometric Imaging Plate Reader as above but stored in the dark at room temperature for 60 min prior to challenge with agonist. The IC₅₀ values were similar after both 3 and 60 min incubation periods and the data were combined. Cells were challenged with a final concentration of 15 nM capsaicin (applied at 10-fold the final concentration in 20 μL added to 180 μL at a velocity of 20 μL/sec) and the fluorescence counts were captured following agonist addition at a sampling rate of 1 Hz for the first 25 sec and 0.33 Hz for another 90 sec. The concentration of capsaicin used in these studies (15 nM) was ˜EC₈₀ for the human recombinant VR1 in this system. The contents of the wells were mixed 3 times (40 μL mix volume) immediately after the additions were made. The saline buffer used for these experiments contained (in mM): 130 NaCl, 2 KCl, 1 MgCl₂, 2 CaCl₂, 20 HEPES pH 7.4. Concentration dependence of block was determined by exposing each well of cells in duplicate rows of a 96 well plate to increasing concentrations of antagonist in half log increments. Column 11 cells were exposed to 30 μM (final concentration) compound. Column 10 cells were exposed to 10 μM (final concentration) compound. Each of these concentrations was made in eppendorf tubes from 10 mM stock solutions in DMSO (at 9-fold the final concentration (see above)) and added to the compound plate (Greiner V-bottom 96 well plate). One hundred eighty (180) μL of the buffer was added to all the other wells. The remaining dilutions were made serially using an 8-channel pipettor and transferring 20 μL of solution from column 11 into column 9, 20 μL of solution from column 10 into column 8, 20 μL of solution from column 9 into column 7, and so forth. The contents of the wells were triturated after transfer of compound. Pipette tips were exchanged after each transfer to avoid carry-over. The magnitude of the capsaicin response was determined by measuring the peak and the final level after 1.5 min exposure to capsaicin. The lower of these values was used to calculate the IC₅₀ value. Data were analyzed using a non-linear regression program (PRISM™ software, GraphPad Software, San Diego, Calif.).

### [³H] Resiniferatoxin Binding Assay

Cell membranes were prepared by washing cells with Hank's Balanced Salt Solution. Cells were dissociated with cell dissociation buffer (Sigma), and then centrifuged at 1000×g for 5 min. Cell pellets were homogenized in cold 20 mM HEPES buffer, pH 7.4, containing 5.8 mM NaCl, 320 mM sucrose, 2 mM MgCl₂, 0.75 mM CaCl₂ and 5 mM KCl and centrifuged at 1000×g for 15 min. The resultant supernatant was then centrifuged at 4000×g for 15 min. The pellet membranes were stored at −80° C. The binding assay procedure was modified from what has been described previously (Szallasi and Blumberg, 1993). Briefly, about 120 μg protein/mL membranes were incubated with the indicated concentration of [³H] RTX (New England Nuclear) in 0.5 mL of the HEPES buffer (pH 7.4) containing 0.25 mg/mL fat acid free bovine serum albumin at 37° C. for 60 min and then the reaction mixture was cooled to 4° C. α₁-Acid glycoprotein (0.1 mg) was added to each sample and was incubated at 4° C. for 15 min. The samples were centrifuged at 18,500 g for 15 min. The tip of the microcentrifuge tube containing the pellets was cut off. The non-specific binding was tested in the presence of 200 nM unlabeled RTX. Bound radioactivity was quantified by scintillation counting. Kᵢ is calculated based on a non-linear regression program (PRISM™ software, GraphPad Software, San Diego, Calif.).

TABLE 2
Vanilloid In vitro assay data

| Ex | IC₅₀ (nM) | Kᵢ (nM) |
|---|---|---|
| 1 | 25, 25, 36 | 267 |
| Comparative A | 120 | 1020 |

1. A VR1 antagonist having the general formula: 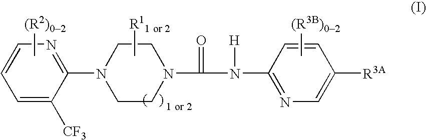
wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═, >NH or >N(C₁₋₄alkyl) and optionally having one unsaturated bond in the ring),
where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONR^{a}R^{b},
or alternatively,
two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R² is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R^{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl,
where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{y})R^{z}, —(C═O)N(R^{y})R^{z}, —(N—R^{t})COR^{t}, —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{p})R^{q}—(N—R^{s})COR^{s} (wherein R^{s} is —H or —C₁₋₆alkyl), —(N—R^{s})SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q}, —(C═O)N(R^{p})R^{q}, (N—R^{s})COR^{s}, —(N—R^{s})SO₂C₁₋₆alkyl, —(C═O)C₁₋₆alkyl, —(C═O)phenyl, —(S═(O)₀₋₂)-C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl;
or a stereoisomer or pharmaceutically acceptable salt, ester, amide or prodrug thereof.

2. The compound of claim 1 wherein R¹ is selected from the group consisting of: —H, methyl, ethyl, n-propyl, i-propyl, n-butyl, i-butyl, t-butyl, n-pentyl, pent-2-yl, hexyl, hex-2-yl, ethenyl, allyl, ethynyl, prop-2-ynyl, cyclopentyl, cyclohexyl, cycloheptyl, trifluoromethyl, pentafluoroethyl, septafluoro-n-propyl, septafluoro-1-propyl, nonafluoro-n-butyl, nonafluoro-1-butyl, nonafluoro-t-butyl, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl and homopiperidin-1-yl,
wherein said methyl, ethyl, n-propyl, i-propyl, n-butyl, i-butyl, t-butyl, n-pentyl, pent-2-yl, hexyl, hex-2-yl, ethenyl, allyl, ethynyl and prop-2-ynyl primary substituents, are optionally mono-substituted with a substituent selected from the group consisting of —F, —Cl, —Br, —I, cyclopentyl, cyclohexyl, -cycloheptyl, trifluoromethyl, pentafluoroethyl, septafluoro-n-propyl, septafluoro-i-propyl, nonafluoro-n-butyl, nonafluoro-i-butyl, nonafluoro-t-butyl, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —(C═O)CH₃, —(C═O)CH₂CH₃, —(C═O)CH₂CH₂CH₃, —(C═O)CH(CH₃)₂, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl and —(C═O)homopiperidin-1-yl.

3. The compound of claim 1 wherein R¹ is selected from the group consisting of: —H, methyl, ethyl, n-propyl, i-propyl, n-butyl, i-butyl and t-butyl, optionally mono-substituted with halo.

4. The compound of claim 1 wherein R¹ is —H or methyl.

5. The compound of claim 1 wherein R¹ is attached to a carbon atom attached to the urea nitrogen.

6. The compound of claim 1 wherein two R¹ are taken together to form a bridging group, and the preferred bridging group is selected from the group consisting of —CH₂— and —CH₂CH₂—.

7. The compound of claim 1 wherein two R¹ are taken together to form a bridging group, and the carbon ring members that are bridged are separated by two ring members.

8. The compound of claim 1 wherein R² are nonexistent or are independently selected from the group consisting of: methyl, ethyl, n-propyl, i-propyl, n-butyl, i-butyl, t-butyl, n-pentyl, pent-2-yl, hexyl, hex-2-yl, ethenyl, allyl, ethynyl, prop-2-ynyl, phenyl, —O-methyl, —O-ethyl, —O-n-propyl, —O-i-propyl, —O-n-butyl, —O-i-butyl, —O-t-butyl, —O-n-pentyl, —Opent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2, 3 or 4-yl, tetrahydrothiopyran-2, 3 or 4-yl, piperidin-2, 3 or 4-yl, N(C₁₋₄alkyl)piperidin-2, 3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-i-propyl, nonafluoro-n-butyl, nonafluoro-i-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl, wherein said methyl, ethyl, n-propyl, i-propyl, n-butyl, i-butyl, t-butyl, n-pentyl, pent-2-yl, hexyl, hex-2-yl, ethenyl, allyl, ethynyl and prop-2-ynyl primary substituents, are optionally mono-substituted with a substituent selected from the group consisting of phenyl, —O-methyl, —O-ethyl, —O-n-propyl, —O-i-propyl, —O-n-butyl, —O-i-butyl, —O-t-butyl, —O-n-pentyl, —O-pent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2,3 or 4-yl, tetrahydrothiopyran-2,3 or 4-yl, piperidin-2,3 or 4-yl, N(C₁₋₄alkyl)piperidin-2,3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-i-propyl, nonafluoro-n-butyl, nonafluoro-i-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl.

9. The compound of claim 1 wherein R² are nonexistent or are selected from the group consisting of —NO₂, —CF₃, —Cl, —F, —CH₃, —CN, —NH₂, —N(CH₃)₂, —OCH₃, tetrahydropyranyl, —CN, —NO₂ and —SO₂NH₂.

10. The compound of claim 1 wherein R^{3A} and R^{3B} are independently selected from the group consisting of: methyl, ethyl, n-propyl, i-propyl, n-butyl, i-butyl, t-butyl, n-pentyl, pent-2-yl, hexyl, hex-2-yl, ethenyl, allyl, ethynyl, prop-2-ynyl, phenyl, —O-methyl, —O-ethyl, —O-n-propyl, —O-i-propyl, —O-n-butyl, —O-i-butyl, —O-t-butyl, —O-n-pentyl, —O-pent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2, 3 or 4-yl, tetrahydrothiopyran-2, 3 or 4-yl, piperidin-2, 3 or 4-yl, N(C₁₋₄alkyl)piperidin-2, 3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-i-propyl, nonafluoro-n-butyl, nonafluoro-i-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl,
wherein said methyl, ethyl, n-propyl, i-propyl, n-butyl, i-butyl, t-butyl, n-pentyl, pent-2-yl, hexyl, hex-2-yl, ethenyl, allyl, ethynyl and prop-2-ynyl primary substituents, are optionally mono-substituted with a substituent selected from the group consisting of phenyl, —O-methyl, —O-ethyl, —O-n-propyl, —O-i-propyl, —O-n-butyl, —O-i-butyl, —O-t-butyl, —O-n-pentyl, —O-pent-2-yl, —O-hexyl, —O-hex-2-yl, —O-phenyl, —O-benzyl, cyclopentyl, cyclohexyl, cycloheptyl, —O-cyclopentyl, —O-cyclohexyl, —O-cycloheptyl, tetrahydropyran-2, 3 or 4-yl, tetrahydrothiopyran-2, 3 or 4-yl, piperidin-2, 3 or 4-yl, N(C₁₋₄alkyl)piperidin-2,3 or 4-yl, tetrahydrofuran-2 or 3-yl, tetrahydrothiophen-2 or 3-yl, pyrrolidin-2 or 3-yl, N(C₁₋₄alkyl)pyrrolidin-2 or 3-yl, —OH, —CN, —NO₂, —NH₂, —NHCH₃, —N(CH₃)₂, —N(CH₂CH₃)₂, —NCH₃(CH(CH₃)₂), imidazolidin-1-yl, 2-imidazolin-1-yl, pyrazolidin-1-yl, piperidin-1-yl, 2- or 3-pyrrolin-1-yl, 2-pyrazolinyl, morpholin-4-yl, thiomorpholin-4-yl, piperazin-1-yl, pyrrolidin-1-yl, homopiperidin-1-yl, —CONH₂, —CONHCH₃, —CON(CH₃)₂, —CON(CH₂CH₃)₂, —CONCH₃(CH(CH₃)₂), —(C═O)imidazolidin-1-yl, —(C═O)-2-imidazolin-1-yl, —(C═O)pyrazolidin-1-yl, —(C═O)piperidin-1-yl, —(C═O)(2- or 3-pyrrolin-1-yl), —(C═O)-2-pyrazolinyl, —(C═O)morpholin-4-yl, —(C═O)thiomorpholin-4-yl, —(C═O)piperazin-1-yl, —(C═O)pyrrolidin-1-yl, —(C═O)homopiperidin-1-yl, —NHCOH, —NHCOCH₃, —NHCOCH₂CH₃, —NHCOCH(CH₃)₂, —N(CH₃)COH, —N(CH₃)COCH₃, —N(CH₃)COCH₂CH₃, —N(CH₃)COCH(CH₃)₂, —NHSO₂CH₃, —NHSO₂CH₂CH₃, —NHSO₂CH(CH₃)₂, —N(CH₃)SO₂CH₃, —N(CH₃)SO₂CH₂CH₃, —N(CH₃)SO₂CH(CH₃)₂, —(C═O)CH₃, —(C═O)CH₂CH₃, —(C═O)CH₂CH₂CH₃, —(C═O)CH(CH₃)₂, —(C═O)phenyl, —SCH₃, —SCH₂CH₃, —SCH₂CH₂CH₃, —SCH(CH₃)₂, —SOCH₃, —SOCH₂CH₃, —SOCH₂CH₂CH₃, —SOCH(CH₃)₂, —SO₂CH₃, —SO₂CH₂CH₃, —SO₂CH₂CH₂CH₃, —SO₂CH(CH₃)₂, —SO₂NH₂, —SO₂NHCH₃, —SO₂N(CH₃)₂, —SO₂N(CH₂CH₃)₂, —SO₂NCH₃(CH(CH₃)₂), —(SO₂)imidazolidin-1-yl, —(SO₂)-2-imidazolin-1-yl, —(SO₂)pyrazolidin-1-yl, —(SO₂)piperidin-1-yl, —(SO₂)(2- or 3-pyrrolin-1-yl), —(SO₂)-2-pyrazolinyl, —(SO₂)morpholin-4-yl, —(SO₂)thiomorpholin-4-yl, —(SO₂)piperazin-1-yl, —(SO₂)pyrrolidin-1-yl, —(SO₂)homopiperidin-1-yl, —SCF₃, —F, —Cl, —Br, —I, trifluoromethyl, pentafluoroethyl, septafluoro-n-propyl, septafluoro-1-propyl, nonafluoro-n-butyl, nonafluoro-1-butyl, nonafluoro-t-butyl, —O-trifluoromethyl, —O-pentafluoroethyl, —O-septafluoro-n-propyl, —O-septafluoro-i-propyl, —O-nonafluoro-n-butyl, —O-nonafluoro-i-butyl, —O-nonafluoro-t-butyl, —COOH, —COO-methyl, —COO-ethyl, —COO-n-propyl, —COO-i-propyl, —COO-n-butyl, —COO-i-butyl, —COO-t-butyl, —COO-n-pentyl, —COO-pent-2-yl, —COO-hexyl and —COO-hex-2-yl.

11. The compound of claim 1 wherein R^{3A} and R^{3B} are independently selected from the group consisting of —CF₃, —OCF₃, butyl, i-propyl, t-butyl, cyclohexyl, cyclopentyl, tetrahydropyranyl, piperidin-1-yl,
1-cyano-1-methylethyl, 2-methoxy-1,1-dimethylethyl, bromo, chloro, fluoro, iodo, methyl, methoxy, nitro, benzyl, 1-trifluoromethylethenyl,
1-trifluoromethylethyl, but-2-yl, benzoyl, nonafluoro-t-butyl and septafluoro-1-propyl.

12. The compound of claim 1 wherein R^{3A} is trifluoromethyl.

13. The compound of claim 1 wherein R^{3B} is nonexistent.

14. The compound of claim 1 wherein said pharmaceutically acceptable salt is an effective carboxylate addition salt.

15. The compound of claim 14 wherein said pharmaceutically acceptable carboxylate addition salt is selected from the group consisting of sodium, potassium, calcium and magnesium.

16. The compound of claim 1 wherein said pharmaceutically acceptable salt is an effective amine addition salt.

17. The compound of claim 16 wherein said pharmaceutically acceptable amino addition salt is selected from the group consisting of hydrobromic, hydroiodic, hydrochloric, perchloric, sulfuric, maleic, fumaric, malic, tartatic, citric, benzoic, mandelic, methanesulfonic, hydroethanesulfonic, benzenesulfonic, oxalic, pamoic, 2-naphthalenesulfonic, p-toluenesulfonic, cyclohexanesulfamic, and saccharic.

18. The compound of claim 1 wherein said pharmaceutically acceptable ester is selected from the group consisting of p-methoxybenzyloxycarbonyl, 2,4,6-trimethylbenzyloxycarbonyl, 9-anthryloxycarbonyl, CH₃SCH₂COO—, tetrahydrofur-2-yloxycarbonyl, tetrahydropyran-2-yloxycarbonyl, fur-2-uloxycarbonyl, benzoylmethoxycarbonyl, p-nitrobenzyloxycarbonyl, 4-pyridylmethoxycarbonyl, 2,2,2-trichloroethoxycarbonyl, 2,2,2-tribromoethoxycarbonyl, t-butyloxycarbonyl, t-amyloxycarbonyl, diphenylmethoxycarbonyl, triphenylmethoxycarbonyl, adamantyloxycarbonyl, 2-benzyloxyphenyloxycarbonyl, 4-methylthiophenyloxycarbonyl, or tetrahydropyran-2-yloxycarbonyl.

19. The compound of claim 1 selected from the group consisting of:

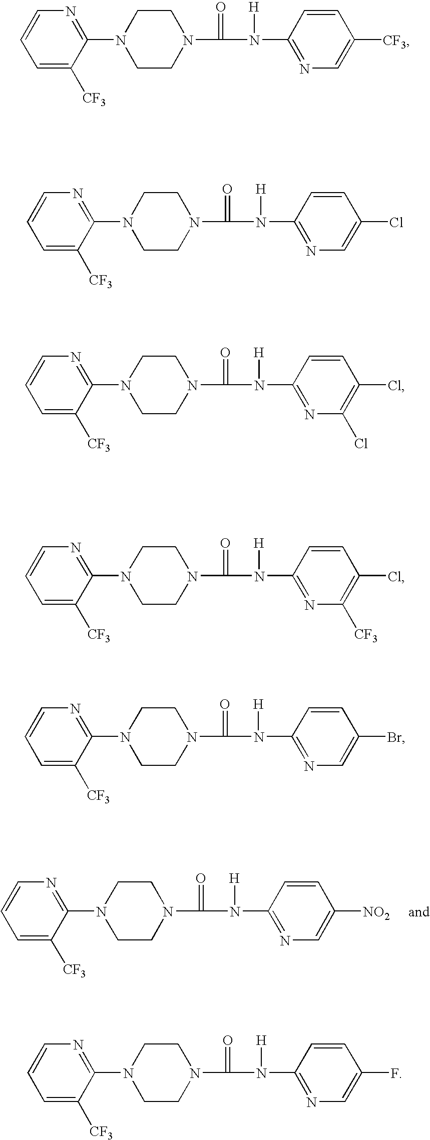

20. The compound of claim 1 which is

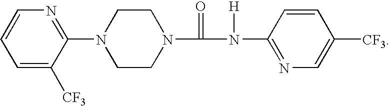

21. A pharmaceutical composition comprising a pharmaceutically acceptable carrier and a therapeutically effective amount of compound having VR1 antagonist activity of formula (I): 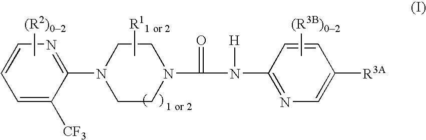
wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═. >NH or >N(C₁₋₄alkyl) and optionally having one unsaturated bond in the ring),
where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONR^{a}R^{b},
or alternatively,
two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R² is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R^{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)-C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl,
where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{y})R^{z}, —(C═O)N(R^{y})R^{z}, —(N—R^{t})COR^{t}, —(N—R^{t})SO₂C₁₋₆alkyl, —(C═O)C₁₋₆alkyl, —(C═O)phenyl, —(S═(O)₀₋₂)-C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{p})R^{q}, —(N—R^{s})COR^{s} (wherein R^{s} is —H or —C₁₋₆alkyl), —(N—R^{s})SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q}, —(C═O)N(R^{p})R^{q}, —(N—R^{s})COR^{s}, —(N—R^{s})SO₂C₁₋₆alkyl, —(C═O)C₁₋₆alkyl, —(C═O)phenyl, —(S═(O)₀₋₂)—C₁₋₆alkyl, SO₂N(R^{p})R^{q}, —SCF₃, halo, perhaloC₁₋₄alkyl, perhaloC₁₋₄alkoxy, —COOH and —COOC₁₋₆alkyl;
or a stereoisomer or pharmaceutically acceptable salt, ester, amide or prodrug thereof.

22. A method for the treatment or prevention of acute or chronic pain or itch in mammals comprising the step of administering to a mammal suffering there from a therapeutically effective amount of compound having VR1 antagonist activity of formula (I): 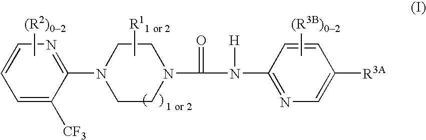
wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═, >NH or >N(C₁₋₄alkyl) and optionally having one unsaturated bond in the ring),
where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONR^{a}R^{b},
or alternatively,
two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R² is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R^{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{y})R^{z}, —(C═O)N(R^{y})R^{z}, —(N—R^{t})COR^{t}, —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{p})R^{q}, —(N—R^{s})COR^{s} (wherein R^{s} is —H or —C₁₋₆alkyl), —(N—R^{s})SO₂C₁₋₆alkyl —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q}, —(C═O)N(R^{p})R^{q}, —(N—R^{s})COR^{s}, —(N—R^{s})SO₂C₁₋₆alkyl, —(C═O)C₁₋₆alkyl, —(C═O)phenyl, —(S═(O)₀₋₂)—C₁₋₆alkyl, —SO₂N(Rₚ)R^{q}, —SCF₃, halo, perhaloC₁₋₄alkyl, perhaloC₁₋₄alkoxy, —COOH and —COOC₁₋₆alkyl;
or a stereoisomer or pharmaceutically acceptable salt, ester, amide or prodrug thereof.

23. A method of claim 22 for said treatment or prevention of acute or chronic pain arising from conditions selected from the group consisting of: osteoarthritis, rotator cuff disorders, rheumatoid arthritis, inflammatory arthritis, fibromyalgia, cluster headache, migraine, headache, sinus headache, tension headache, toothache, burn, sunburn, dermatitis, psoriasis, eczema, insect sting or bite, bony fractures, ligamentous sprains, plantar fasciitis, costochondritis, tendonitis, bursitis, tennis elbow, pitcher's elbow, patellar tendonitis, repetitive strain injury, myofascial syndrome, muscle strain, myositis, temporomandibular joint disorder, stump pain, low back strain, neck strain, whiplash, bladder spasms, interstitial cystitis, urinary tract infection, ureteral colic, renal colic, pharyngitis, cold sores, stomatitis, external otitis, otitis media, burning mouth syndrome, mucositis, esophageal pain, gastroesophageal reflux, pancreatitis, enteritis, irritable bowel disorder, inflammatory bowel disease, Crohn's disease, ulcerative colitis, diverticulosis, diverticulitis, hemorrhoids, anal fissures, proctitis, rectal pain, cholecystitis, labor, childbirth, intestinal gas, abdominal pain, menstrual cramps, pelvic pain, vulvodynia, vaginitis, testicular pain, pleurisy, pericarditis, contusions, abrasions, peripheral neuropathy, diabetic neuropathy, acute herpetic neuralgia, post-herpetic neuralgia, trigeminal neuralgia, glossopharyngeal neuralgia, atypical facial pain, causalgia, reflex sympathetic dystrophy, sciatica, cervical, thoracic or lumbar radiculopathy, brachial plexopathy, lumbar plexopathy, phantom limb pain, occipital neuralgia, intercostal neuralgia, supraorbital neuralgia, inguinal neuralgia, meralgia paresthetica, genitofemoral neuralgia, carpal tunnel syndrome, Morton's neuroma, post-mastectomy syndrome, post-thoracotomy syndrome, post-polio syndrome, Guillain-Barré syndrome, Raynaud's syndrome, coronary artery spasm (Printzmetal's or variant angina), esophageal spasm, osteolytic metastases, osteoblastic metastases, primary bone cancer, cancerous invasion of bone, visceral cancer pain, neuropathic cancer pain, Paget's disease and multiple myeloma.

24. A method of claim 22 for said treatment or prevention of itching arising from dermatological or inflammatory conditions selected from the group consisting of: renal or hepatobiliary disorders, immunological disorders, medication reactions and unknown/idiopathic conditions.

25. A method for the treatment or prevention of inflammation in mammals comprising the step of administering to a mammal suffering there from a therapeutically effective amount of compound having VR1 antagonist activity of formula (I): 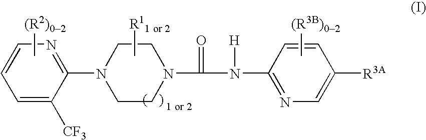
wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═. >NH or >N(C₁₋₄alkyl) and optionally having one unsaturated bond in the ring),
where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONR^{a}R^{b},
or alternatively,
two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R² is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R^{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl,
where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{y})R^{z}, —(C═O)N(R^{y})R^{z}, —(N—R^{y})COR^{t}, —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{p})R^{q}, —(N—R^{s})COR^{s} (wherein R^{s} is —H or —C₁₋₆alkyl), —(N—R^{s})SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q}, —(C═O)N(R^{p})R^{q}, —(NR^{s})COR^{s}, —(N—R^{s})SO₂C₁₋₆alkyl, —(C═O)C₁₋₆alkyl, —(C═O)phenyl, —(S═(O)₀₋₂)-C₁₋₆alkyl, SO₂N(R^{p})R^{q}, —SCF₃, halo, perhaloC₁₋₄alkyl, perhaloC₁₋₄alkoxy, —COOH and —COOC₁₋₆alkyl;
or a stereoisomer or pharmaceutically acceptable salt, ester, amide or prodrug thereof.

26. A method of claim 25 for said treatment or prevention of inflammatory manifestations of diseases and conditions selected from the group consisting of: inflammatory bowel disease (ulcerative colitis and Crohn's disease) psoriasis and psoriatic arthritis, rheumatoid arthritis, myasthenia gravis, multiple sclerosis, scleroderma, glomerulonephritis, pancreatitis, inflammatory hepatitis, asthma, chronic obstructive pulmonary disease, allergic rhinitis, uveitis and cardiovascular manifestations of inflammation including atherosclerosis, myocarditis, pericarditis and vasculitis.

27. A method for the treatment or prevention of gastrointestinal and urinary tract disorders in mammals comprising the step of administering to a mammal suffering there from a therapeutically effective amount of compound having VR1 antagonist activity of formula (I): 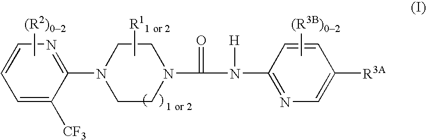
wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═. >NH or >N(C₁₋₄alkyl) and optionally having one unsaturated bond in the ring),
where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONR^{a}R^{b},
or alternatively,
two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R² is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R^{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl,
where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{y})R^{z}, —(C═O)N(R^{y})R^{z}, —(N—R^{t})COR^{t}, —(N—R^{t})SO₂C₁₋₆alkyl, —(C═O)C₁₋₆alkyl, —(C═O)phenyl, —(S═(O)₀₋₂)-C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{p})R^{q}, —(N—R^{s})COR^{s} (wherein R^{s} is —H or —C₁₋₆alkyl), —(N—R^{s})SO₂C₁₋₆alkyl —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q}, —(C═O)N(R^{p})R^{q}, —(NR^{s})COR^{s}, —(N—R^{s})SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl;
or a stereoisomer or pharmaceutically acceptable salt, ester, amide or prodrug thereof.

28. A method of claim 27 for said treatment or prevention of gastrointestinal and urinary tract disorders selected from the group consisting of: nausea, vomiting, intestinal cramping, intestinal bloating, bladder spasms, urinary urgency, defecation urgency and urge incontinence.

29. A method for the treatment or prevention of tracheobronchial and diaphragmatic dysfunction in mammals comprising the step of administering to a mammal suffering there from a therapeutically effective amount of compound having VR1 antagonist activity of formula (I): 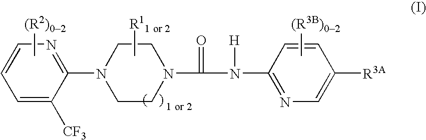
wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═. >NH or >N(C₁₋₄alkyl) and optionally having one unsaturated bond in the ring),
where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONR^{a}R^{b},
or alternatively,
two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R² is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R^{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl,
where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R_{y})R_{z}, —(C═O)N(R^{y})R^{z}, —(N—R^{t})COR^{t}, —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{p})R^{q}, —(N—R^{s})COR^{s} (wherein R^{s} is —H or —C₁₋₆alkyl), —(N—R^{s})SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₁₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q}, —(C═O)N(R^{p})R^{q}, —(N—R^{s})COR^{s}, —(N—R^{s})SO₂C₁₋₆alkyl, —(C═O)C₁₋₆alkyl, —(C═O)phenyl, —(S═(O)₀₋₂)—C₁₋₆alkyl, SO₂N(R^{p})R^{q}, —SCF₃, halo, perhaloC₁₋₄alkyl, perhaloC₁₋₄alkoxy, —COOH and —COOC₁₋₆alkyl;
or a stereoisomer or pharmaceutically acceptable salt, ester, amide or prodrug thereof.

30. A method of claim 29 for said treatment or prevention of tracheobronchial and diaphragmatic dysfunction associated with conditions selected from the group consisting of: cough, asthma, bronchospasm, chronic obstructive pulmonary disease, chronic bronchitis, emphysema and hiccups (hiccoughs, singultus).
